(19) United States
(12) Patent Application Publication
(10) Pub. No.: US 2008/0248591 A1
(43) Pub. Date: Oct. 9, 2008

(54) Gas bubble biosensor

(76) Inventor: Alan Joseph Bauer, Jerusalem (IL)

Correspondence Address: Dr. Alan Joseph Bauer, 49 Ussishkin, Jerusalem, IL, 94542, US

(21) Appl. No.: 11/892,324

(22) Filed: Aug. 22, 2007

Related U.S. Application Data
(60) Provisional application No. 60/839,321, filed on Aug. 22, 2006.

(51) Int. Cl. G01N 33/543; B01J 19/00

(52) U.S. Cl. 436/518; 422/83

(57) ABSTRACT

The present invention describes a biosening device and method. Specifically, binding of target analyte perturbs the surface of a sensor strip so that gas bubbles are generated in solution. Said gas bubbles may be detected for determination of analyte presence in sample.

### BACKGROUND OF THE INVENTION

1. Field of the Invention

This invention pertains to a sensor and method for detecting or quantifying analytes. More particularly the present invention is directed to the detection of analytes analyte-responsive gas bubble generation during analyte interaction with a immobilized binding agents on a sensor strip.

2. Description of the Related Art

Chemical and biological sensors are devices that can detect or quantify analytes by virtue of interactions between targeted analytes and macromolecular binding agents such as enzymes, receptors, DNA strands, heavy metal chelators, or antibodies. Such sensors have practical applications in many areas of human endeavor. For example, biological and chemical sensors have potential utility in fields as diverse as blood glucose monitoring for diabetics, detection of pathogens commonly associated with spoiled or contaminated food, genetic screening, and environmental testing.

Chemical and biological sensors are commonly categorized according to two features, namely, the type of material utilized as binding agent and the means for detecting an interaction between binding agent and targeted analyte or analytes. Major classes of biosensors include enzyme (or catalytic) biosensors, immunosensors and DNA biosensors. Chemical sensors make use of synthetic macromolecules for detection of target analytes. Some common methods of detection are based on electron transfer, generation of chromophores, or fluorophores, changes in optical or acoustical properties, or alterations in electric properties when an electrical signal is applied to the sensing system.

Enzyme (or catalytic) biosensors utilize one or more enzyme types as the macromolecular binding agents and take advantage of the complementary shape of the selected enzyme and the targeted analyte. Enzymes are proteins that perform most of the catalytic work in biological systems and are known for highly specific catalysis. The shape and reactivity of a given enzyme limit its catalytic activity to a very small number of possible substrates. Enzymes are also known for speed, working at rates as high as 10,000 conversions per second per enzyme molecule. Enzyme biosensors rely on the specific chemical changes related to the enzyme/analyte interaction as the means for determining the presence of the targeted analyte. For example, upon interaction with an analyte, an enzyme may generate electrons, a colored chromophore or a change in pH (due to release of protons) as the result of the relevant catalytic enzymatic reaction. Alternatively, upon interaction with an analyte, an enzyme may cause a change in a fluorescent or chemiluminescent signal that can be recorded by an appropriate detection system.

Immunosensors utilize antibodies as binding agents. Antibodies are protein molecules that bind with specific foreign entities, called antigens, which can be associated with disease states. Antibodies attach to antigens and either remove the antigens from a host and/or trigger an immune response. Antibodies are quite specific in their interactions and, unlike enzymes, they are capable of recognizing and selectively binding to very large bodies such as single cells. Thus, antibody-based biosensors allow for the identification of certain pathogens such as dangerous bacterial strains. As antibodies generally do not perform catalytic reactions, there is a need for special methods to record the moment of interaction between target analyte and recognition agent antibody. Changes in mass (surface plasmon resonance, acoustic sensing) are often recorded; other systems rely on fluorescent probes that give signals responsive to interaction between antibody and antigen. Alternatively, an enzyme bound to an antibody can be used to deliver the signal through the generation of color or electrons; the enzyme-linked immunosorbent assay (ELISA) is based on such a methodology.

DNA biosensors utilize the complementary nature of the nucleic acid double-strands and are designed for the detection of DNA or RNA sequences usually associated with certain bacteria, viruses or given medical conditions. A sensor generally uses single-strands from a DNA double helix as the binding agent. The nucleic acid material in a given test sample is then denatured and exposed to the binding agent. If the strands in the test sample are complementary to the strands used as binding agent, the two interact. The interaction can be monitored by various means such as a change in mass at the sensor surface or the presence of a fluorescent or radioactive signal. Alternative arrangements provide binding of the sample of interest to the sensor and subsequent treatment with labeled nucleic acid probes to allow for identification of the sequences of interest.

Chemical sensors make use of non-biological macromolecules as binding agents. The binding agents show specificity to targeted analytes by virtue of the appropriate chemical functionalities in the macromolecules themselves. Typical applications include gas monitoring or heavy metal detection; the binding of analyte may change the conductivity of the sensor surface or lead to changes in charge that can be recorded by an appropriate field-effect transistor (FET). Several synthetic macromolecules have been used successfully for the selective chelation of heavy metals such as lead.

The present invention has applicability to all of the above noted binding agent classes.

Known methods of detecting interaction of analyte and binding agent can be grouped into several general categories: chemical, optical, acoustical, electrical, and electrochemical. In the last, a voltage or current is applied to the sensor surface or an associated medium. As binding events occur on the sensor surface, there are changes in electrical properties of the system. The leaving signal is altered as function of analyte presence.

While hundreds of sensors have been described in patents and in the scientific literature, actual commercial use of such sensors remains limited. In particular, virtually all sensor designs set forth in the prior art contain one or more inherent weaknesses. Some lack the sensitivity and/or speed of detection necessary to accomplish certain tasks. Other sensors lack long-term stability. Still others cannot be sufficiently miniaturized to be commercially viable or are prohibitively expensive to produce. Some sensors must be pre-treated with salts and/or enzyme cofactors, a practice that is inefficient and bothersome. To date, virtually all sensors are limited by the known methods of determining that contact has occurred between an immobilized binding agent and targeted analytes. Use of fluorescent or other external detection probes adds to sensor production requirements and reduces lifetimes of such sensor systems. Additionally, the inventor believes that there is no sensor method disclosed in the prior art that is generally applicable to the vast majority of macromolecular binding agents, including non-redox enzymes, antibodies, antigens, nucleic acids, receptors, and synthetic binding agents.

### SUMMARY OF THE INVENTION

It is therefore a primary object of some aspects of the present invention to provide an improved analyte detection system, in which a sensor strip composed of a base member and a binding agent layer is used in the detection of analyte through the generation of analyte-responsive gas bubbles.

It is a further object of some aspects of the invention to describe an optical detection system for gas bubbles generated by interaction of analyte with said sensor strip.

It is a still further object of some aspects of the invention to describe a detection system for gas bubbles that are physically located on a container in which sample and sensor strip reside.

It is an additional object of some aspects of the invention to improve the consistency and ease of use in detection of an analyte in a sensor system by performing biosensing in an optically clear disposable container such as a plastic cuvette or test tube.

It is yet an additional object of some aspects of the invention to improve the consistency and ease of use in detection of an analyte in a sensor system by performing biosensing in the presence of hydrogen peroxide for non-enzymatic generation of analyte-responsive gas bubbles.

The practice of the present invention does not require application of an external electrical signal, enzyme-based oxidation-reduction detection schemes, or the presence of an active electrode in an aqueous solution. Furthermore, the present invention does not rely on arrays or changes of applied electrical fields or signals as a function of analyte presence. In some embodiments, the present invention may be practiced in the absence of hydrogen peroxide.

The methodology of analyte detection described herewith is very sensitive. Using the method of the present invention, it is possible to detect specific pathogenic bacteria consistently in a complex matrix within three minutes at 20 cells per milliliter of sample.

In general, measurement of analyte-responsive gas bubbles according to the present invention allows for simple, rapid, specific, inexpensive and sensitive determination of analyte presence. Methods for detecting analyte-related gas bubbles in solution include but are not limited to optical and imaging methodologies. In some embodiments, a detection unit may be employed to detect optical or other signals associated with analyte-responsive gas bubble generation. In other embodiments, appearance of gas bubbles can be determined visually in the absence of any detection unit. These latter embodiments are particularly useful in low-technology settings as in the detection of malaria or meningitis in third-world countries. In embodiments in which hydrogen peroxide is present, peroxide presence additionally allows for sterilization of sample prior to the latter's disposal.

A sensor strip according to some aspects of the invention may contain a plurality of identical or unique sensor strips so as to increase system detection redundancy and/or multiple analyte detection capabilities. Component binding agent layers of a composite sensor strip may be individually monitored, each component strip forming a part of a single sensor strip.

In embodiments of the invention sensor strips are prepared from a portion of a container in which a biosensing experiment is performed. In such a case, binding agents specific for analyte are immobilized in proximity to a portion of the container in which sample of interest is added. Peroxide, generally hydrogen peroxide (H₂O₂), may optionally be added to sample prior to exposing sample to sensor strip in the container. Final H₂O₂ concentrations should be less than 1% (volume to volume, v:v), but concentrations higher than 10% v:v have been successfully tested on different analytes. Optimal H₂O₂ concentration is 0.3% v:v final concentration in sample. The present invention may be practiced in the absence of hydrogen peroxide, with analyte binding causing gas bubble formation through analyte-responsive precipitation of dissolved gas.

As analyte presence leads to presence of gas bubbles in solution, several methods are available for detecting the generated gas bubbles, the number of which is roughly related to the quantity of analyte in sample. In some cases, gas bubbles may be visualized directly on the container in which biosensing occurs. Alternatively, gas bubbles may be visualized directly on the sensor strip, when a sensor strip is separate from the container used in biosensing. Gas bubble detection may also be effected by the bubbles' scattering effect on light shown either on said container or on the sensor strip (when a separate element) itself. Alternatively, images of samples may be processed to identify the presence of analyte-responsive gas bubbles in sample container. Secondary phenomena related to gas formation such as solution convection or dissolved gas concentration/partial pressure may alternatively be monitored.

The invention provides a sensor for detecting an analyte, which minimally includes a base member, a binding agent layer associated with the base member and a gas bubble detector. The base member and the binding agent layer minimally define a sensor strip, while additional layers such as a protective packaging layer over the binding agents may be included in the term “sensor strip” if they are physically associated with the base member. Analyte presence is correlated to gas bubbles present in solution after interaction of a sample of interest with sensor strip.

One aspect of the sensor has sensor strip exposed to hydrogen peroxide during or after sensor strip exposure to sample.

Another aspect of the sensor has sample heated prior to sample exposure to sensor strip.

Still an additional aspect of the sensor has sample transiently exposed to high pressure gas prior to sample exposure to sensor strip.

An aspect of the sensor includes a chemical entity bound to the base member and disposed proximate the binding agent layer.

Yet another aspect of the sensor includes a container in which sensor strip and sensor strip are present during biosensing.

One aspect of the sensor includes a packaging layer disposed above the binding agent layer. The packaging layer is soluble in a medium that contains the analyte.

According to another aspect of the sensor, analyte presence is correlated to gas bubbles in said container. Said gas bubbles may be visually detected or may be identified by their perturbation or scattering of light directed at said container.

According to a further aspect of the sensor, analyte presence is correlated to changes in optical images of sample prior to and after sample addition.

According to a further aspect of the sensor, analyte presence is correlated to the affect of analyte-responsive gas bubbles on a Doppler ultrasound signal.

According to another aspect of the sensor, the analyte is a plurality of unique analytes for detection.

In another aspect of the sensor, said base member may actually be a portion of said container, with binding agents bound either directly or through the agency of a chemical layer to said portion of said container.

In still another aspect of the sensor, an inhibitor to the enzyme catalase is added to the sample prior to sample exposure to hydrogen peroxide, when employed.

The invention provides a method for detecting a predetermined analyte, including the steps of providing a base member, and forming a binding agent layer of macromolecules in proximity to the base member surface, wherein the macromolecules are capable of interacting at a level of specificity with the predetermined analyte. The method further includes steps of exposing said sample to said sensor strip, and detecting gas bubbles in said container.

One aspect of the method has the further step of binding a chemical entity to the base member and forming the binding agent layer proximate the chemical entity.

Another aspect of the method as the further step of exposing said sensor strip to hydrogen peroxide at a final concentration of 0.3% volume to volume, during or after sensor strip has been exposed to sample.

An aspect of the method includes detecting said gas bubble through visual observation or perturbation of light directed at said container.

An aspect of the method includes monitoring perturbations in light directed at said container holding sample as a function of gas bubble formation in said container.

In another aspect of the method, detecting analyte involves analyzing changes in optical images of sample to detect gas bubble presence.

In still another aspect of the method, multiple analytes are detected through the agency of a single or multiple sensor strips.

In a further aspect of the method, a portion of the container serves as the base member for binding agent layer formation.

One aspect of the method includes disposing a packaging layer above the binding agent layer. The packaging layer is soluble in a medium that contains the predetermined analyte.

According to another aspect of the method, the sensor strip includes a plurality of sensor strips.

According to yet another aspect of the method, sample is activated immediately prior to sensor strip exposure to said sample.

The invention further provides a sensor for detecting an analyte, which minimally includes a base member, a binding agent layer associated with the base member, hydrogen peroxide at concentration of 0.3%. The base member and the binding agent layer minimally define a sensor strip, while additional layers such as a packaging layer over the binding agents may be included in the term “sensor strip” if they are physically associated with the base member. Analyte presence is corrected to gas bubbles present after interaction of a sample containing hydrogen peroxide with sensor strip.

### BRIEF DESCRIPTION OF THE DRAWINGS

For a better understanding of these and other objectives of the present invention, reference is made to the following detailed description of the invention, by way of example, which is to be read in conjunction with the following drawings, in which like elements differ by multiples of 100, and wherein:

FIG. 1 is a schematic view of a sensor detection system 100, which is constructed and operative in accordance with an embodiment of the invention, wherein a sensor strip 122 comprised of base member 120, chemical entity 130, binding agent layer 140 and packaging layer 150 rests in sample 180 in clear plastic container 185;

FIG. 2 is a schematic of a sensor detection system 200 that is constructed and operative in accordance with an alternate embodiment of the invention, wherein a portion of a clear plastic container 285 serves as the base member 220 on which binding agent layer 240 is constructed;

FIG. 3 is a schematic of a sensor detection system 300 that is constructed and operative in accordance with an alternate embodiment of the invention, wherein a disposable plastic container 385 is analyzed for gas bubbles 399 on its surface by an external light source 395;

FIG. 4 is a schematic of a sensor detection system 400 that is constructed and operative in accordance with an alternate embodiment of the invention, wherein an optical imaging apparatus 496 is used to image bubbles 499 generated through analyte 457 binding;

FIG. 5 is a schematic of a sensor detection system 500 that is constructed and operative in accordance with an alternate embodiment of the invention. The sensor detection system 500 is similar to the sensor detection system 100 (FIG. 1), and like elements have like reference numerals, advanced by 400. In this sensor detection system 500, an ultrasound device 592 is used to detect analyte-responsive gas bubbles 599 in sample 580.

FIG. 6 is a schematic of a sensor detection system 600 that is constructed and operative in accordance with an alternate embodiment of the invention. The sensor detection system 600 is similar to the sensor detection system 100 (FIG. 1), and like elements have like reference numerals, advanced by 500. In this sensor detection system 500, a light source 697 and light detector 698 are used for the detection of bubbles 699 in solution.

FIG. 7 is a schematic of a sensor detection system 700 that is constructed and operative in accordance with an alternate embodiment of the invention. The sensor detection system 700 is similar to the sensor detection system 600 (FIG. 6), and like elements have like reference numerals, advanced by 100. In this sensor detection system 700, a light source 797 and light detector 798 are used for the detection of bubbles 799 on sensor strip 522 base member 520.

FIG. 8 is a photograph of an experiment performed with sensor strips corresponding to sensor detection system 100 (FIG. 1) in which sensor strips were used for the unique detection of a specific bacterial target in accordance with a disclosed embodiment of the invention.

FIG. 9 is a photograph of an experiment performed with sensor strips corresponding to sensor detection system 400 (FIG. 4) in which sensor strips were used for the unique detection of a specific bacterial target in accordance with a disclosed embodiment of the invention.

FIG. 10 is a photograph of results for detection of a pathogenic bacteria in a meat sample in accordance with a disclosed embodiment of the invention.

FIG. 11 is a photograph of overnight colony growths for the experiment whose results are shown in FIG. 10, in accordance with a disclosed embodiment of the invention.

### DETAILED DESCRIPTION OF THE INVENTION

In the following description, numerous specific details are set forth in order to provide a thorough understanding of the present invention. It will be apparent, however, to one skilled in the art that the present invention may be practiced without these specific details. In other instances well-known circuits and control logic have not been shown in detail in order not to unnecessarily obscure the present invention.

### Definitions

Certain terms are now defined in order to facilitate better understanding of the present invention.

An “analyte” is a material that is the subject of detection or quantification.

A “base member” is a solid element on which binding agents are immobilized. The term “base member” refers to a solid material on which binding agents are physically immobilized. Base members may be conductive or insulating in their electrical properties. “Macromolecules”, “macromolecular binding agents”, “binding agents” or “macromolecular entities” can be any natural, mutated, synthetic, or semi-synthetic molecules that are capable of interacting with a predetermined analyte or group of analytes at a level of specificity.

A “binding agent layer” is a layer composed of one or a plurality of binding agents. The binding agent layer may be composed of more than one type of binding agent. A binding agent layer may additionally include molecules other than binding agents. Crosslinking agents may be applied to bind separate components of a binding agent layer together.

A “chemical entity” is a chemical layer that is disposed proximate a base member either one or both sides of the base member. The chemical entity rests between the base member and the binding agent layer. The chemical entity serves to immobilize binding agents proximate base member. Chemical entities may be differentially deposited on opposite sides of a base member surface by any means or multiple layers on a given side of the base member may be considered a single chemical entity.

A “packaging layer” is defined as a chemical layer disposed above the binding agent layer. The packaging layer may aid in long term stability of the macromolecules, and in the presence of a sample that may contain analyte of interest, the packaging layer may dissolve to allow for rapid interaction of analyte and binding agents.

A “sensor strip” is defined as a minimum of a single base member and its associated binding agent layer. The base member surface and any macromolecular entities, chemical entities, packaging layers or other elements physically associated with the base member are included in the term “sensor strip”.

A “peroxide” refers to any material of structure R—O—O—R′. In hydrogen peroxide, R═R′=hydrogen. The expression “peroxide” refers to hydrogen peroxide and other members of this class of chemicals. “Degradation” with respect to hydrogen peroxide refers specifically to the breakdown of hydrogen peroxide to water and oxygen gas. “Dissolved oxygen” has its normal meaning in the art and refers to oxygen dissolved in a solution and is generally reported in ppm. “Activation”, “activating” and “activated” with regard to the present invention refers to the optional process of imparting energy to sample immediately prior to sensor strip interaction with sample. Activation may be performed my mixing, stirring, heating, centrifugation, shaking, or the like.

A “gas bubble” has its normal meaning, referring to a thin, usually spherical or hemispherical film of liquid filled with air or gas A “gas bubble detector” refers to a device that can identify or quantify bubbles in a container with a liquid sample. “Catalase inhibitor” refers to a chemical that inhibits the enzyme catalase and thus prevents its catalytic degradation of hydrogen peroxide to oxygen and water.

Without being bound by any particular theory, the following discussion is offered to facilitate understanding of the invention. The sensor design disclosed herein is based on analyte-responsive generation of gas bubbles in an aqueous solution. The sensor utilizes a novel method of detecting an analyte wherein macromolecular binding agents are first immobilized as a binding agent layer proximate a solid base member. Base member may be any solid material to which binding agents may be directly or indirectly tethered. Binding of analyte causes thermodynamic changes, whose net impact is to cause dissolved gas to leave solution in the form of gas bubbles. To date, oxygen released from hydrogen peroxide has been the gas of choice for saturating sample solution, though other gases such as nitrogen and carbon dioxide could also be used. Increasing dissolved gas prior to biosensing can be performed through changes in sample temperature or through treatment of sample with pressurized gas. In some aspects of the present invention, the advantages of particular forms of sensor strip embodiments are disclosed. Specifically, a sensor strip may be a separate element of base member and binding agents or alternatively may be formed directly as part of a container in which a biosensing experiment according to aspects of the present invention is performed.

### First Embodiment

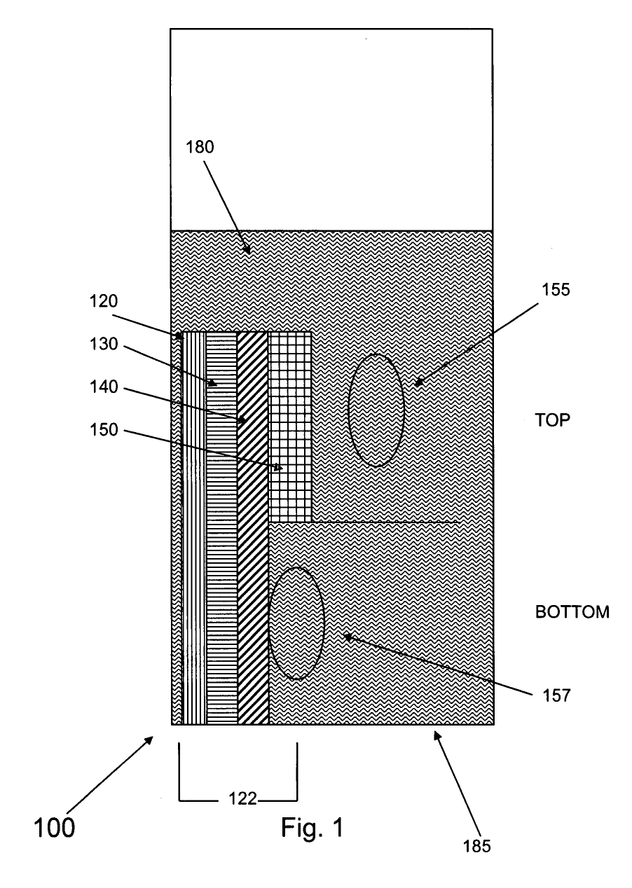

Reference is now made to FIG. 1, which is a schematic of a sensor detection system 100 that is constructed and operative in accordance with an embodiment of the invention. Container 185 holds sample 180 that contains un-bound analyte (TOP, 155) and hydrogen peroxide, H₂O₂ (not shown) at a concentration of greater than 0.1% (volume:volume) but not in excess of 10% (volume:volume). A sensor strip 122 composed of solid base member 120, chemical entity 130, binding agent layer 140 and packaging layer 150 is present in the container 185 when sample 180 is added. The packaging layer 150 dissolves (BOTTOM, FIG. 1) to allow for binding of analyte (157, bound analyte). Bound analyte 157 leads to precipitation of dissolved gas. Gas bubbles may be detected by several known means such as Total Internal Reflection or through optical image analysis software.

The packaging layer 150, shown on the TOP of FIG. 1, is a layer of water-soluble chemicals deposited above the immobilized macromolecules of the binding agent layer 140. The packaging layer 150 may be deposited by soaking or spraying methods. The packaging layer 150 serves to stabilize the binding agent layer 140 during prolonged dry storage. In the absence of a packaging layer, oil and dirt may build up on the hydrophilic binding agent layer 140 and may interfere with the rapid action of the sensor system. A commercial solution, StabilGuard (Surmodics, Inc., 9924 West 74^{th} Street, Eden Prairie, Minn., 55344, USA) is typically used for the packaging layer 150 so as to guarantee packaging layer dissolution in aqueous samples, and thus facilitate direct interaction between macromolecular binding agents of binding agent layer 140 and analytes 157. Other chemicals may be chosen for use in the packaging layer. Water-soluble polymers, sugars, salts, organic, and inorganic compounds are all appropriate for use in preparation of the packaging layer 150.

As shown on the TOP of FIG. 1, free analyte 155 is disposed proximate the packaging layer 150 prior to the latter's dissolution. When the packaging layer 150 dissolves, the macromolecules incorporated in the binding agent layer 140 are free to immediately interact with analyte 157, as shown on the BOTTOM of FIG. 1. After dissolution of the packaging layer 150, analyte 157 is shown interacting with the binding agent layer 140 on the BOTTOM of FIG. 1. The analyte 155, 157 can be a member of any of the following categories, listed herein without limitation: cells, organic compounds, antibodies, antigens, virus particles, pathogenic bacteria, toxins, metals, metal complexes, ions, spores, yeasts, molds, cellular metabolites, enzyme inhibitors, receptor ligands, nerve agents, peptides, proteins, fatty acids, steroids, hormones, narcotic agents, synthetic molecules, medications, enzymes, nucleic acid single-stranded or double-stranded nucleic acid polymers. The analyte 155 can be present in a solid, liquid, gas or aerosol. The analyte 155 could even be a group of different analytes, that is, a collection of distinct molecules, macromolecules, ions, organic compounds, viruses, toxins, spores, cells or the like that are the subject of detection or quantification. Some of the analyte 157 physically interacts with the sensor strip 122 after dissolution of the packaging layer 150 and causes an increase in gas bubbles present in solution. There is no requirement for application of a voltage or other electrical signal to the sensor strip 122 prior to or during biosensing.

Examples of macromolecular binding agents suitable for use as the binding agent layer 140 include, but are not limited to non-redox enzymes that recognize substrates and inhibitors, antibodies that bind antigens, antigens that recognize target antibodies, receptors that bind ligands, ligands that bind receptors, nucleic acid single-strand polymers that can bind to form DNA-DNA, RNA-RNA, or DNA-RNA double strands, and synthetic molecules that interact with targeted analytes. The present invention can be practiced with non-redox enzymes, peptides, proteins, antibodies, antigens, catalytic antibodies, fatty acids, receptors, receptor ligands, nucleic acid strands, as well as synthetic macromolecules as the binding agents in the binding agent layer 140. Natural, synthetic, semi-synthetic, over-expressed and genetically-altered macromolecules may be employed as binding agents. The binding agent layer 140 may form monolayers, multilayers or mixed layers of several distinct binding agents or binding agents with other chemical components (not shown). A monolayer of mixed binding agents may also be employed (not shown). The binding agents in the binding agent layer 140 may be cross-linked together with glutaraldehyde or other chemical cross-linking agents.

The macromolecule component of the binding agent layer 140 is neither limited in type nor number. Non-redox enzymes, peptides, receptors, receptor ligands, antibodies, catalytic antibodies, antigens, cells, fatty acids, synthetic molecules, and nucleic acids are possible macromolecular binding agents in the present invention. The sensor detection system 100 may be applied to detection of many classes of analyte because it relies on the following properties shared by substantially all applications and embodiments of the sensor detection system according to the present invention:

(1) that the macromolecules chosen as binding agents are highly specific entities designed to bind only with a selected analyte or group of analytes;

(2) that analytes may interact at a level of specificity with the macromolecules;

(3) that binding of analyte with binding agent causes ion release; and

(4) that the ion release can lead to the precipitation of gas dissolved in solution. This gas release event can most readily be detected by the presence of gas bubbles in solution. These gas bubbles generally stick to the surface of the sensor strip used for biosensing or on the walls sample container. The gas bubbles may be detected on the strip, in sample or attached to the container in which biosensing occurs.

In order to increase the energy of sample components prior to biosensing, the sample may be activated. Activation is generally performed by rapidly mixing sample with a “vortex” mixer or the like. Alternatively, the sample may be shaken, heated, centrifuged or otherwise treated so as to increase the kinetic energy of the sample components immediately prior to sensor strip exposure to sample. After activation, sensor strip is placed in sample and gas bubble detection begins.

The broad and generally applicable function of the sensor detection system 100 is preserved during formation of the binding agent layer 140 in proximity to the base member 120 because the binding agent layer 140 formation can be effected by either specific covalent attachment or general physical absorption. A chemical entity 130, such as a self-assembled monolayer, may be used in the physical absorption of the binding agent layer 140 proximate the base member 120. It is to be emphasized that the catalytic degradation of hydrogen peroxide that is associated with analyte presence does not depend on any specific enzyme chemistries, optical effects, fluorescence, chemiluminescence or applied electrical signals. These features are important advantages of the present invention. Additionally, hydrogen peroxide kills pathogenic samples during biosensing.

### Second Embodiment

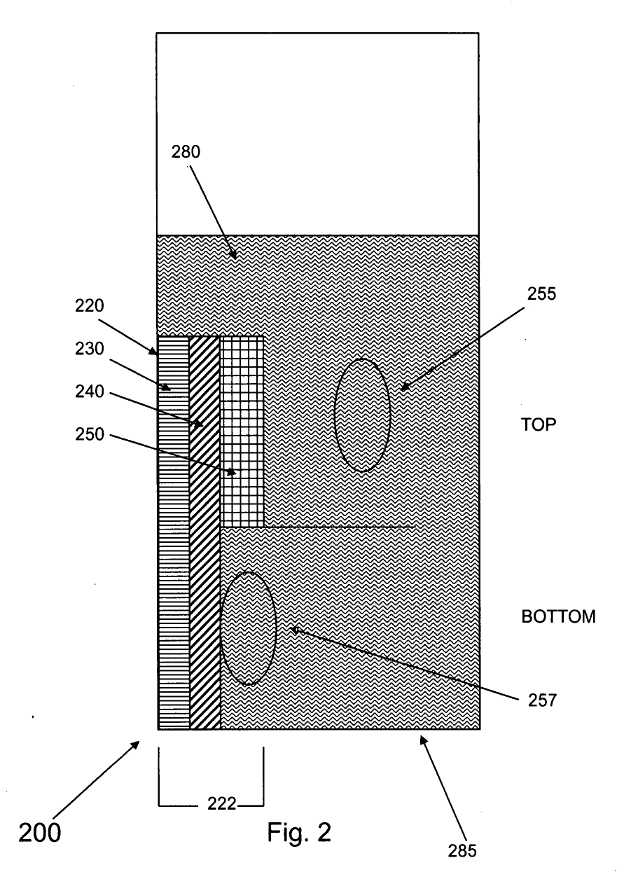

Reference is now made to FIG. 2, which is a schematic of a an alternative embodiment of a sensor detection system 200 that is constructed and operative in accordance with an embodiment of the invention. Container 285 holds sample 280 that contains un-bound analyte (TOP, 255) and hydrogen peroxide, H₂O₂ (not shown) at a concentration of greater than 0.1% (volume:volume) but not in excess of 10% (volume:volume). A sensor strip 222 composed a base member 220 made from a portion of the container 285, optional chemical entity 230, binding agent layer 240 and packaging layer 250 is present in the container 285 when sample 280 is added. The packaging layer 250 dissolves (BOTTOM, FIG. 2) to allow for binding of analyte (257, bound analyte).

Binding of analyte 257 leads to ion release that causes dissolved gas to coalesce in the form of gas bubbles. The gas bubbles may be detected by several means, as discussed previously.

### Third Embodiment

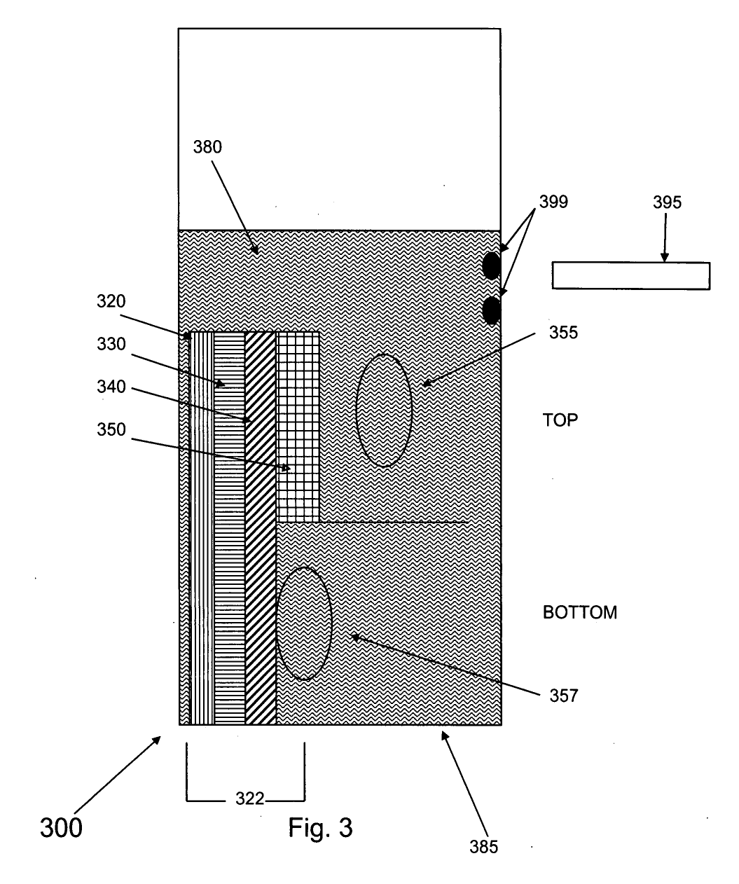

Reference is now made to FIG. 3, which is a schematic of an alternative embodiment of a sensor detection system 300 that is constructed and operative in accordance with an embodiment of the invention. Container 385 holds sample 380 that contains un-bound analyte (TOP, 355) in solution. A sensor strip 322 composed of solid base member 320, chemical entity 330, binding agent layer 340 and packaging layer 350 is present in the container 385 when sample 380 is added. The packaging layer 350 dissolves (BOTTOM, FIG. 3) to allow for binding of analyte (357, bound analyte). Bound analyte 357 evolves gas that appear in the form of gas bubbles 399. The gas bubbles are detected as gas bubbles 399 on the walls of container 385 containing sample 380.

### Fourth Embodiment

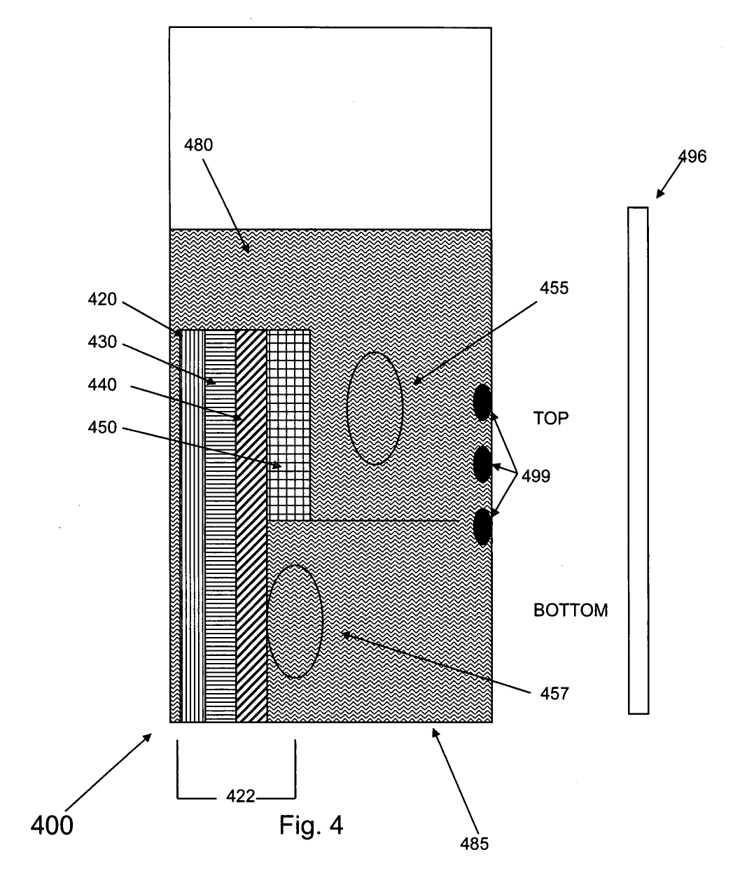

Reference is now made to FIG. 4, which is a schematic of an alternative embodiment of a sensor detection system 400 that is constructed and operative in accordance with an embodiment of the invention. Container 485 holds sample 480 that contains un-bound analyte (TOP, 455) and prior to biosensing, sample 480 was cooled to increase dissolved gas and then returned to room temperature (process not shown). A sensor strip 422 composed of solid base member 420, chemical entity 430, binding agent layer 440 and packaging layer is present in the container 485 when sample 480 is added. The packaging layer 450 dissolves (BOTTOM, FIG. 4) to allow for binding of analyte (457, bound analyte). Binding of analyte 457 leads to increased precipitation of dissolved gas. Gas bubbles 499 are detected by an imaging gas bubble detector 496. The imaging device may be a digital camera modified with image analysis software for gas bubble 499 detection. Gas bubbles 499 leave a unique imprint on images of the sample 480 taken by gas bubble detector 496.

### Fifth Embodiment

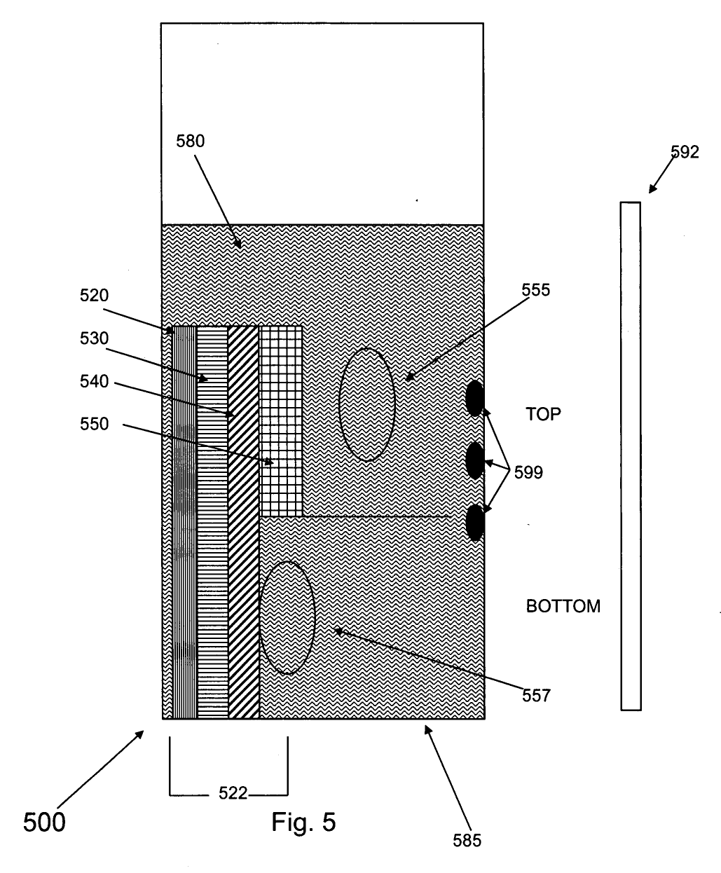

Reference is now made to FIG. 5, which is a schematic of an alternative embodiment of a sensor detection system 500 that is constructed and operative in accordance with an embodiment of the invention. Container 585 holds sample 580 that contains un-bound analyte (TOP, 555) and hydrogen peroxide, H₂O₂ (not shown) at a concentration of greater than 0.001% (volume:volume) but not in excess of 10% (volume:volume). A sensor strip 522 composed of plastic base member 520, optional chemical entity 530, binding agent layer 540 and packaging layer 550 is present in the container 585 when sample 580 is added. The packaging layer 550 dissolves (BOTTOM, FIG. 5) to allow for binding of analyte 557, bound analyte. Bound analyte 557 leads to increased gas bubble 599 presence in container. An ultrasound device gas bubble detector 592 is used to detect the gas bubbles 599 in solution.

### Sixth Embodiment

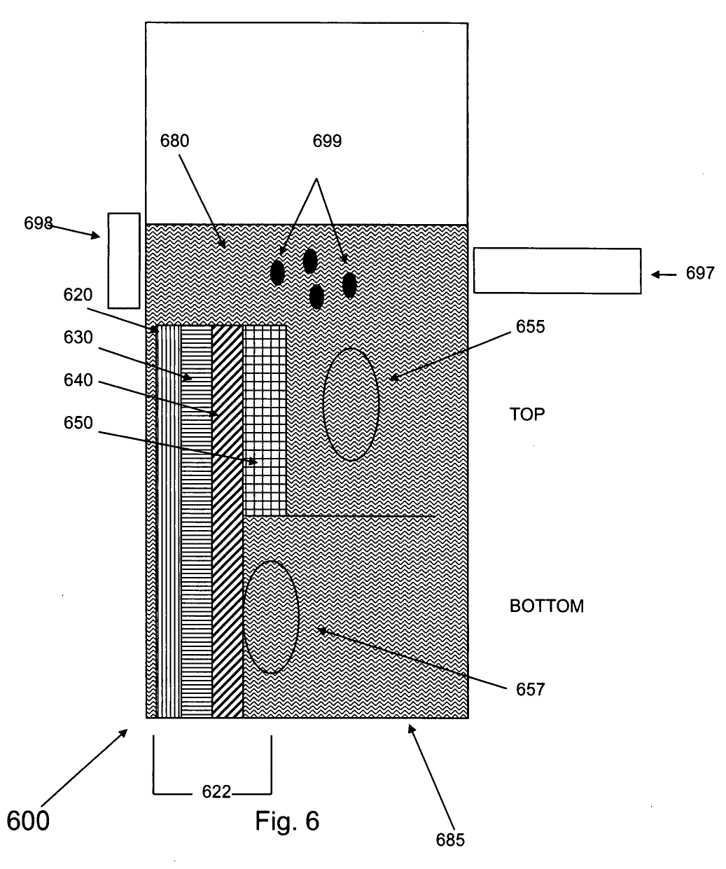

Reference is now made to FIG. 6, which is a schematic of an alternative embodiment of a sensor detection system 600 that is constructed and operative in accordance with an embodiment of the invention. Optically clear container 685 holds sample 680 that contains un-bound analyte (TOP, 655) and buffered hydrogen peroxide, H₂O₂ (not shown) at a concentration of greater than 0.1% (volume:volume) but not in excess of 10% (volume:volume). A sensor strip 622 composed of solid base member 620, chemical entity 630, binding agent layer 640 and packaging layer is present in the container 685 when sample 680 is added. The packaging layer 650 dissolves (BOTTOM, FIG. 6) to allow for binding of analyte (657, bound analyte). Bound analyte 657 leads to evolution of gas in the form of gas bubbles. Gas bubbles 699 are detected by the interference of gas bubbles 699 with light propagated from a gas bubble detector light source 697 to a light detector 698.

### Seventh Embodiment

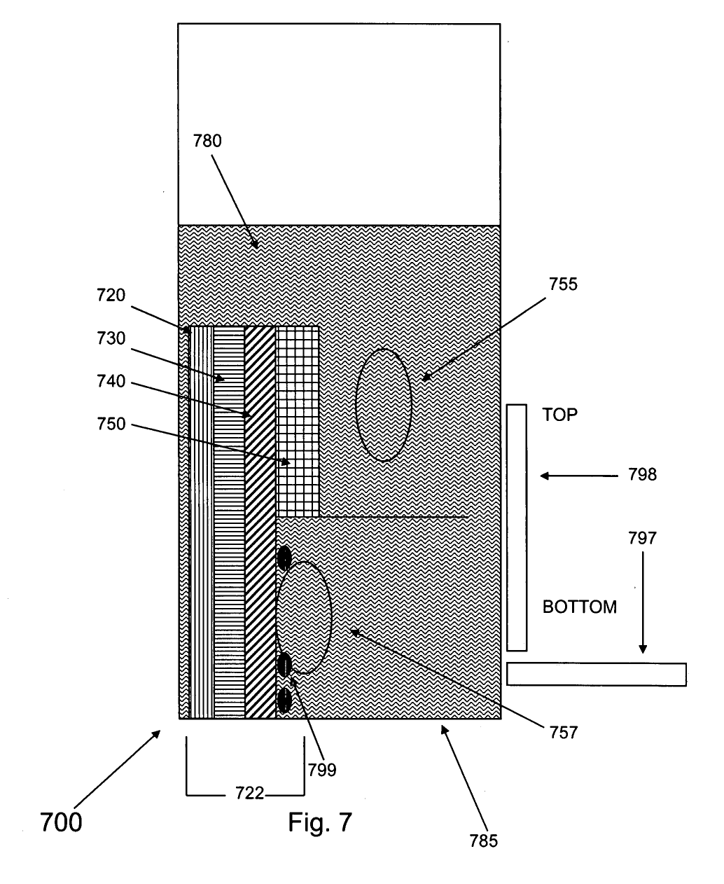

Reference is now made to FIG. 7, which is a schematic of an alternative embodiment of a sensor detection system 700 that is constructed and operative in accordance with an embodiment of the invention. Optically clear container 785 holds sample 780 that contains un-bound analyte (TOP, 755) and hydrogen peroxide, H₂O₂ (not shown) at a concentration of greater than 0.1% (volume:volume) but not in excess of 10% (volume:volume). A sensor strip 722 composed of solid base member 720, chemical entity 730, binding agent layer 740 and packaging layer is present in the container 785 when sample 780 is added. The packaging layer 750 dissolves (BOTTOM, FIG. 7) to allow for binding of analyte (757, bound analyte). Bound analyte 757 extrudes electric double layer ions and thus causes precipitation of dissolved gas. Gas bubbles 799 are detected by their effect on the optical properties of light propagated from a light source 797, reflected off the sensor strip 722 base member 720 and measured in a gas bubble detector 798 as shown in FIG. 7.

### Example 1

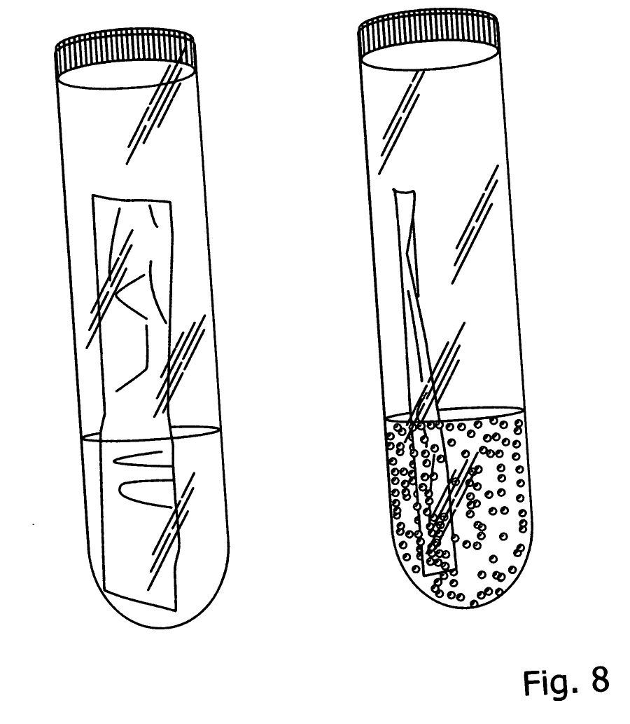

The analysis in this example was performed using the embodiment of FIG. 1. Testing for Pseudomonas aeruginosa was performed in phosphate buffer solution, pH 7.15. Aluminum foil having a matte surface and a shiny surface (Extra Heavy-Duty Diamond Foil, Reynolds Metals Co., 555 Guthridge Court, Norcross, Ga. 30092) was cut into 6 centimeter by 8 centimeter pieces and soaked in an ethanolic (Carmel Mizrahi, Rishon Letzion, Israel, 95%) solution of docosanoic acid (21,694-1, Aldrich Chemical Company, Milwaukee, Wis.) for 20 minutes and then rinsed with distilled water. The soakings were performed in 100 milliliter piranha-treated (70% sulfuric acid; 30% hydrogen peroxide) beakers, with the self-assembled monolayer (SAM) surfactant solution standing at 20 milliliters in the beaker. Hydrophobic SAM-coated foil pieces were rinsed in deionized water and next transferred to 20 milliliters of aqueous phosphate-buffered solutions (pH 7.2) of polyclonal antibodies specific for P. aeruginosa antigen (Product B47578P, Biodesign International, 60 Industrial Park Road, Saco, Me. 04072 USA) at an approximate concentration of 18 microgram per milliliter. The solution was kept in contact with the SAM-coated aluminum foil for approximately 20 minutes and then the coated aluminum foil was rinsed with phosphate buffer lacking antibody. The hydrophilic coated aluminum foil was next soaked for 3 minutes in 20 milliliters of StabilGuard (SG01-0125, Surmodics, 9924 West 74^{th} Street, Eden Prairie, Minn. 55344). After coating, the coated foil was dried at 37 degrees Celsius for approximately one hour, after which it was transferred to a sealed bag that contained calcium sulfate drying agent (238988-454G, “Drierite”, Aldrich Chemical Company). Prior to use, the coated foil was removed from its storage bag. 1 cm×10 cm rectangles of coated sensor strip 122 were cut and placed in plastic test tubes. Samples were prepared from phosphate buffer (8 mM) that contained hydrogen peroxide at 0.1% (v:v). One sample contained Pseudomonas aeruginosa cells at an approximate concentration of 10⁴ cells per milliliter, while the other sample contained E. coli at a similar concentration. Each sample 180 was added to the appropriate container 185 containing the coated sensor strip 122 composed of aluminum base member 120, SAM chemical entity 130, a binding agent layer 140 composed of polyclonal antibodies and StabilGuard packaging layer 150. As shown in FIG. 8, the sample with Pseudomonas aeruginosa (right tube) showed significant gas bubble presence, while the sample that contained the non-target E. coli (left tube) showed no noticeable bubbling.

### Example 2

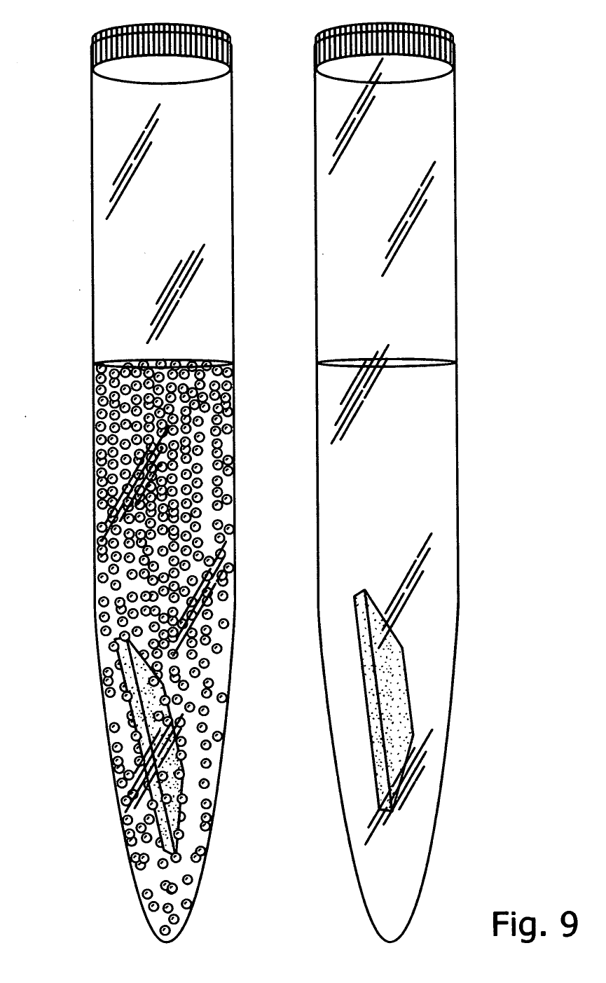

The analysis in this example was performed using the embodiment of FIG. 5. N-type silicon (Silicon Sense, N.H., USA) was cut into 1×1 cm² pieces and rinsed in 95% ethanol (Carmel Mizrahi, Israel). The chips were then rinsed in deionized water and placed in piranha solution. After piranha cleaning for 30 minutes at 80 degrees Celsius, the chips were rinsed in copious amounts of DI water, and then transferred to a 20 milliliter solution of ammonium fluoride (Aldrich product number 338869; 40% weight:volume in DI water). When the chips appeared hydrophobic due to the generation of silicon hydride on the chip surfaces, the chips were transferred to a phosphate-buffered solution of Pseudomonas-specific polyclonal antibodies (Biodesign, Product B47578P) mixed in a 1:100 ratio with bovine serum albumin (BSA, Sigma Chemical Co.). The chips readily became hydrophilic as phosphate and then protein bound to the surface. The chips were next transferred to StabilGuard for packaging layer formation and then allowed to dry at 37 degrees Celsius. In this example, silicon acts as base member 520, phosphate serves as chemical entity 530, polyclonal antibodies with BSA form the binding agent layer 540, while StabilGuard is the packaging layer 550. Dried chips were transferred to samples 580 in Eppendorf tube containers 585 that contained either sample 580 with either Pseudomonas aeruginosa cells (FIG. 9, left side) or E. coli (FIG. 9, right side) in addition to dilute amounts of hydrogen peroxide. A digital camera was used as an imaging device 592 to produce the photographic image shown in FIG. 9. As is clear from the samples shown in FIG. 9, the sample with Pseudomonas analyte 555, 557 shows much greater gas bubble formation than does the sample that lacks analyte recognized by the binding agent layer 540. No catalase was added to the samples described in this example.

### Example 3

P-type silicon (Silicon Sense, N.H., U.S.A.), was scored with a diamond pen and cut into 0.5 cm by 0.5 cm square chips. The chips were placed in a 100-milliliter glass beaker. Two hundred such squares were rinsed in situ sequentially in chloroform, then ethanol and finally in deionized water. Excess water was removed and 60 milliliters piranha solution (70% sulfuric acid; 30% hydrogen peroxide) was added. The chips were left in piranha solution at 80 degrees Celsius for 30 minutes. Solution was decanted, the chips were washed in situ with copious amounts of deionized water, and then treated with 20 milliliters of 40% weight to volume ammonium fluoride (Aldrich, product number 338869, Milwaukee, Wis. U.S.A.). The chips were left in the ammonium fluoride etching solution for twenty minutes. Removal of silicon oxide left the chips hydrophobic and many of them began to float. The solution was carefully decanted and the chips were rinsed with copious amounts of deionized water. The now hydrophobic chips, in the chemical form of Si—H (silicon hydride) were soaked in 20 milliliter potassium phosphate buffer (25 mM, pH 7.5) of bovine serum albumin (BSA, Sigma, 100 micrograms) and antibody ((0.2 micrograms of catalogue sample C65160M, Biodesign, Saco, Me., U.S.A.) for E. coli 0157:H7. The chips became hydrophilic and dropped to the bottom of the beaker. The chips were allowed to soak in protein solution for thirty minutes. Solution was discarded, the chips were rinsed with 25 mM potassium phosphate solution once and then treated with 20 milliliters of Stabilguard (SG01-0125, Surmodics, 9924 West 74^{th} Street, Eden Prairie, Minn. 55344). After ten minutes, Stabilguard was decanted, the chips were poured out onto paper and put into an incubator for fifteen minutes at 37 degrees Celsius to dry. Individual chips were used for experiments as described below.

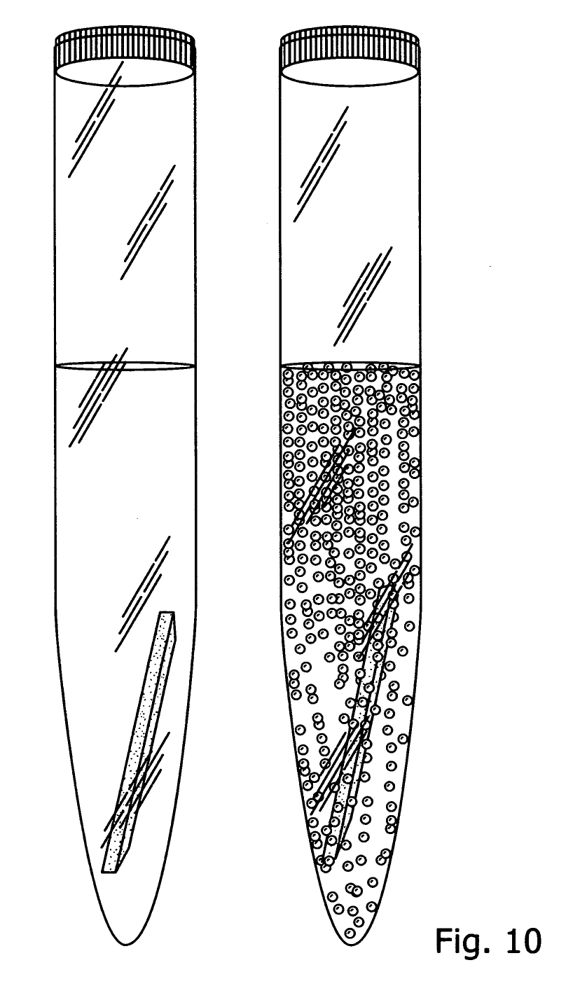

E. coli 0157:H7 (ATCC strain 433894) grown overnight in tryptic-soy growth media (BS-376, Novamed, Jerusalem, Israel) was serially diluted in 10 mM potassium phosphate buffer. A 10⁸ dilution of target bacteria was used. The bacteria were added to a 25 mM potassium phosphate solution (1.5 milliliters) that was 20 microgram per milliliter in catalase (Catalogue number C-40, Sigma). The bacteria were allowed to sit in buffer for two minutes and then hydrogen peroxide (Per-O-Flex, diluted in deionized water tenfold to 0.3% stock) was added to a final concentration of 0.001%. The solution was divided between two Eppendorf tubes. A coated chip was inserted into one of the tubes, while into the second was placed an uncoated silicon chip for the purposes of a control experiment. Thirty seconds after chip insertion bubbles were visible to the eye exclusively in the tube that had the chip coated with antibodies for E. coli 0157:H7. Two minutes later, a photographic image of the experiment was recorded, as shown in FIG. 10.

In this example, based on the sensor detection system 400 embodiment shown in FIG. 4, silicon serves as base member 420 for binding agents 440, with phosphate moieties acting as chemical entity 430 between them. Sensor strip 422 includes silicon chip base member 420, phosphate chemical entity 430, antibody and bovine serum albumin binding agent layer 440 and Stabilguard packaging layer 450. Eppendorf tube serves as container 485 for bacterial sample 480, containing analyte 455, 457 E. coli 0157:H7. A Sony 2.1 megapixel digital camera serves as the optical imaging apparatus 496.

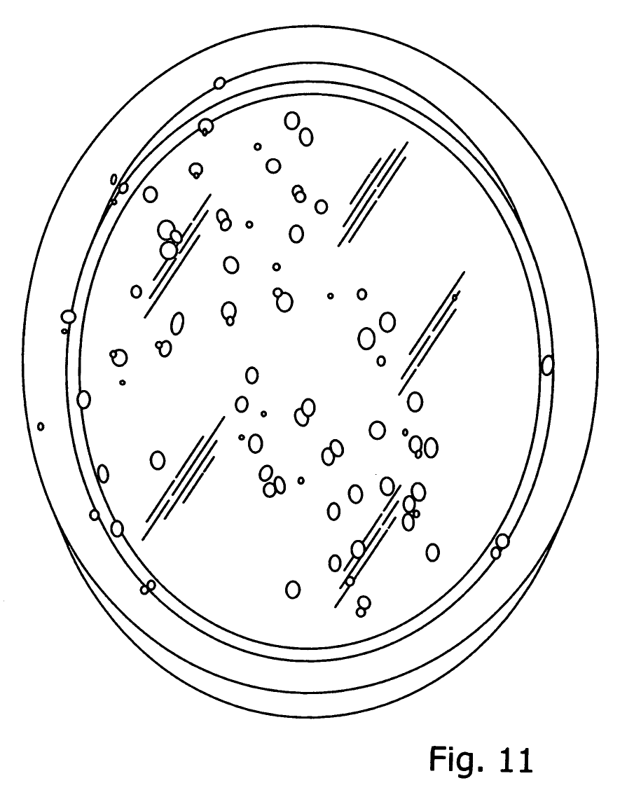

FIG. 10 shows the results for the E. coli 0157:H7 detection experiment described above. The tube on the right side contains coated sensor chip and there are hundreds of bubbles visible in this image of the experiment. Tilting the tube 45 degrees helps in bringing the bubbles to the surface of the Eppendorf tube. Bubbles in positive experiments tend to be small and closely-spaced. Bubble size, bubble position or pattern in container, number of bubbles and/or speed of bubble appearance on container wall may all be considered in discriminating positive from negative samples. The negative control (left tube) showed no bubbling, though it was exposed to the identical solution as was present in the positive sample. Plating of a parallel sample not treated with hydrogen peroxide showed that only a few dozen cells were present in the experiment. Results of plating of the 10⁸ dilution used in this example are shown in FIG. 11.

The present invention may be performed either in a single or in multiple steps. If target is located in a sample that contains significant amounts of catalase, catalase inhibitor may be added to sample or alternatively, sensor strip may first be soaked in sample, then rinsed in deionized water and then soaked in a solution that contains hydrogen peroxide at 0.3% v:v. In the final soak with hydrogen peroxide, gas bubbles are identified as a function of analyte presence in the original sample. The strip may be manually moved between sample, rinse and hydrogen peroxide solutions or may be appropriately handled in a detection unit that automatically changes solution around the sensor strip.

### SUMMARY

The implications of the invention described herein are that nearly any material that can be recognized at a level of specificity by a peptide, protein, antibody, non-redox enzyme, receptor, nucleic acid polymer, synthetic binding agent, or the like can be detected and quantified safely in food, body fluids, air or other samples quickly, cheaply, and with high sensitivity. Response is very rapid, generally less than 10 minutes. Cost of manufacture is low, and sensitivity has been shown to be very good.

The present invention has been described with a certain degree of particularity, however those versed in the art will readily appreciate that various modifications and alterations may be carried out without departing from the spirit and scope of the following claims. Therefore, the embodiments and examples described here are in no means intended to limit the scope or spirit of the methodology and associated devices related to the present invention. Sample may be presented to the sensor strip by static or flow means, including but not limited to microfluidic delivery of sample to sensor strip.

1. A biosensor for detecting or quantifying of an analyte in a sample, comprising:
a base member;
a binding agent layer, wherein said binding agent layer and said base member define a sensor strip, macromolecules of said binding agent layer being interactive at a level of specificity with a predetermined analyte; and,
a gas bubble detector.

2. The sensor according to claim 1, further comprising a chemical entity disposed between said base member and said binding agent layer.

3. The sensor according to claim 1 further comprising a container in which sample and sensor strip are placed.

4. The sensor according to claim 1, further comprising a light source in said bubble detector.

5. The sensor according to claim 1, further comprising an optical detection component of said bubble detector.

6. The sensor according to claim 1, wherein said analyte represents a plurality of unique analytes.

7. The sensor according to claim 1, wherein said base member is physically associated with the container in which sample is placed.

8. The sensor according to claim 1 wherein sensor strip is exposed to hydrogen peroxide during or after sensor strip exposure to sample.

9. A method for detecting a predetermined analyte in a sample, comprising the steps of:
providing a solid base member;
forming a binding agent layer of macromolecules in proximity to said base member, said binding agent layer and said base member defining a sensor strip, wherein said macromolecules are capable of interacting at a level of specificity with said predetermined analyte;
exposing sensor strip to sample; and,
detecting gas bubbles in said container.

10. The method according to claim 9, further comprising the steps of:
binding a chemical entity to a base member surface; and
forming said binding agent layer proximate said chemical entity.

11. The method according to claim 9, further comprising the step of
exposing sensor strip to hydrogen peroxide at a final concentration of 0.3% volume to volume.

12. The method according to claim 9, wherein said gas bubbles are detected visually in said container or on said sensor strip.

13. The method according to claim 9, wherein said gas bubbles are detected through their perturbation of light directed by a gas bubble detector at said container.

14. The method according to claim 9, wherein said gas bubbles are detected by light scattered by said gas bubbles in said container.

15. The method according to claim 9, wherein the base member is a portion of the container into which sample is added.

16. The method according to claim 9, wherein said gas bubbles are detected by means of their perturbation of light transmission through the container.

17. The method according to claim 9, wherein said gas bubbles are detected by their appearance in an optical image of sample taken by a gas bubble detector.

18. The method according to claim 9, wherein said analyte represents a plurality of unique analytes.

19. The method according to claim 9, wherein said sensor strip represents a plurality of sensor strips.

20. An electrode-free biosensor for detection or quantification of an analyte in a sample, comprising:
a base member;
a binding agent layer, wherein said binding agent layer and said base member define a sensor strip, macromolecules of said binding agent layer being interactive at a level of specificity with a predetermined analyte; and,
hydrogen peroxide.
